(19) United States
(12) Patent Application Publication
(10) Pub. No.: US 2005/0080103 A1
(43) Pub. Date: Apr. 14, 2005

(54) Procedure for asthmatic, and other non-invasive surgery for brain tumor cures

(76) Inventor: Frank Gulisano, Brooklyn, NY (US)

Correspondence Address: FRANK GULISANO, APT 2G, 1212 LORING AVENUE, BROOKLYN, NY, 11208-5046, US

(21) Appl. No.: 10/780,128

(22) Filed: May 20, 2004

Related U.S. Application Data
(60) Provisional application No. 60/509,592, filed on Oct. 9, 2003.

(51) Int. Cl. A61K 31/46

(52) U.S. Cl. 514/304

(57) ABSTRACT

My procedual cure explains and identifies the drug and drugs role in bringing about a cure for brain tumors. It is a natural grown drug that has been chemical changed by me. This changed drug now becomes destructive to a tumor's abnormal chemistry, I have included the amount in weight of such a drug, the timing and duration of timing of its use along with other pertinent information so as to make the treatment tailored to each patient for such a cure. Existing medical equipment mention, to be staffed by qualified personel. This procedure does not need invasive surgery or radioactive substances for tumor cures. I have included an alternative procedure in the event my brain procedure does not work on other body tumors or as well, such as metastasis ones. I have explained how such a cure may come about, in correlating this alternative to my original procedure.

I have effected the cure of my asthma that I was born with, which came to an end at approximately 55 years of age, and was done by utilizing mother nature's pure cocain powder in a free base form, the timing and knowledge of its use is medically crucial. The size of this free base piece that I used many years ago was ¼ the size of a cardboard match head, matches that one receives with a pack a cigarettes. I recall this weight to be about 1/10 of 100 parts of a gram or 10/100 on a centimeter scale, of pure 84% free base natural powder cocain. To insure the proper strength dosage is applied per full breath intake once only per session, start with a new prepared metal screen with a new 1/10 of a gram cocain piece installed on this screen that can be evaporated at least twice This needs a very small butane flame to evaporated, as this cocain is now very sensitive to heat, that has cause another chemical change. Air is to be drawn through this evaporation for a full deep breath capacity for that person. When the heat is taken away, this piece instantly stops evaporating. Each metal screen must be burned red hot before using for a few seconds to evaporate the industrial chemical residue on it that goes up in a puff of smoke, then the filter is clean to be used.

Seeing as each person has a different lung capacity, such person's full air capacity must be determine before time, and the amount of air/cocain mixture used, to equal it in volume and must be deeply inhaled one time only per one half hour or another predetermined time, as explained below. This volume measurement must be done before time due to the fact that an a unconscious patient may not take a full breath while unconscious but take small breaths at the beginning of the time settings. The volume of prepared mixed of air and chemically changed cocain should always be used up (which should be about three to four liters of air mixture) to equal that persons deep intake volume. I recall, this tumor cure took me 16 hours or 32 sessions of one half hour apiece of deep inhaling this free base 1/10 of a gram chemical changed cocain, one after another, non stop after the end of each of the one half hours, as its metabolic rate came to an end at this point, like clock work, each and every time. This might have been over done by me, as I stopped due to fatigue. But this was done for a completely different reason in trying to find out information about a different subject, and I did not realized what I did to myself until 4 months later, in my not getting any more asthma attacks. This took me about a year to figure out what happened. Before starting anything on the patient, they must not have any intake of food for at least eight hours before starting anything, unless the physician requests more time. The first step after eight hours or more being in a calm state while awake is to check their blood until there is a steady normal metabolic rate. The second step is to determine the patients full lung capacity. The third step is to put the patient to sleep for as many hours as is needed, for inhaling air/cocain mixture once only at first, so as to check the patients blood at intervals for signs of when there is a complete metabolic use of this changed substance for that person only. This metabolic time frame must be noted exactly and the machines used to calibrated to that time frame for each succeeding full volume of intake of this mixture which is tailored to each patient, so as not to over do this mixture intake or under do it to any one. The fourth step is to continue this procedure until the scanning of the brain shows a decreasing electrical output in a part of the brain as this procedure is continuing, when this brain current stops at any brain point, the tumor was there, and is now dead, but this will take many hours to accomplish. Such tumors can also be located in time by tagging this freebase cocain with radioactive isotopes. The fifth step is to stop everything and at this point, leave the patient to rest naturally, for as long as is needed for them to wake up, as the brain adjusts itself, and will wake up on its own normally. The original cocain C17H2N04 is used in snorting, but when free based with ammonia (not heated) or baking soda (I used baking soda) or ether and heated carefully in a cooking glass and worked, it becomes free of its alkaloid base and is instantly absorbed in the blood, when heated carefully again for evaporation and inhaled. This then breaches the brain barrier in seconds to where the tumor or tumors are located. This amount of alkaloid base is approximately 1/11 of the original amount in weight, and goes into a solution with what you are freebasing it with, so that what is left is 1/11 lighter than what you started with, and can at this point be inhaled and becomes useless in snorting it. It should be noted that cocain is a very corrosive substance to flesh due to its alkaloid content, but when free based this alkaloid substance is eliminated and the chemical composition of cocain has changed, when this is heated for evaporation it goes through its final chemical changes, that tumors have an infinitive attraction to it, in bringing about its demise. An asthmatic attack is caused by a brain tumor in its release of an electrical charge which is erratic at its best, as it has no normal order in behavior when some thing sets it off, that can be caused by its internal action alone, or set off with outside stimuli that gets into the blood stream setting this tumor off directly or indirectly. When this happens the tumor's electrical charge upsets glands it has access to, one in particular whose name and its secretion I do not recall, but this secretion was recently discovered, (due to updated equipment that was then available, which was about 15 years ago) in the blood of people that had asthmatic attacks (no one knew why this gland was doing this, it was just raw findings). This secretion was found to be very minute and powerful and causes the bronchial tubes to constrict and an asthma attacks was imminent. When such bronchial tubes recovered, this person started to breath better. I had my own good reasons to believe it was a tumor causing this problem all along in producing an unwanted electrical current to this gland that would cause it to discharge its brew in a sufficient amount in causing an attack as minute as it was. If a medical scientist had known the underlying reason for such an attack in the past, what could they have done? Besides once an attack occurred it was to late to do anything to prevent it. I thought if the electric charge was very strong at times, it would release an unusual amount of the substance mention above and became too much for a person's body to over come in a safe time, and eventually caused death by asphyxiation. (It has been estimated by health officials, that approximately 5,000 deaths occur each year in the United States that were asthmatic related). This tumor I believe must be near or connected to the breathing apparatus in complicating things. The reason for the following logic is that tumors that are else where must cause different problems for the body, depending on what net work its hooked up to, and is explained below. The only safe way to kill such tumors malignant or not, curable or not is by my method above, and does not need invasive surgery or radioactive substance in doing away with such tumors. Tumors that cause asthmatic attacks or any tumors, I believe are brought about by defective genes in creating these tumors. If such genes can only be pinpointed! perhaps it can be found in working backwards, from the tumor to the gene so as to prevent tumors from forming, by studying such genes. Any abnormal cells in the brain that causes a brain problem is consider a tumor to me and may or may not not be called tumors by the medical profession. This holds true for me for tumors in any part of the body. Pharmaceutical cocain which is man made is harsh and very strong at 100% pure due to its lack of natural cocain derivatives. This may or may not work in killing such tumors when free based, and heated again, if not then the derivatives of natural cocaine plays an important role in a free base state in effecting a cure for such tumors. The reason cocaine is the only substance to do the job that I know of is a simple one, it is the only substance that can breach the brain barrier where these tumors are located, that are sensitive to this new cocain's ingredients, that effectively kill these tumors by self destruct. When tumors absorb this new substance of the finished product, it disrupts it's cell chemistry in its greedy need or use of it as quickly as possible, with no normal restrains, and its intake is over done. Due to the fact a tumor takes most of this new substance normal cells receive very little if any as their chemical composition is different and more stable causing no harm to such normal cells in the time it takes to kill such tumors. To cure tumors by nasal intake, if it can be done with non free base cocain I believe would destroyed your mucous membrane first, and intravenous of such I would think is much to risky. Young abandon children using cocain of every form excessively in Peru became deform, as the brain did not developed right, and could not perform its functions in making healthy organs. Using cocain excessively or not by anyone, especially such young developing bodies is extremely dangerous in its long time use. This became a very big problem In the country of Peru, so that operations were perform that were simple brain procedures, and were successful on such children, in getting rid of their cocain desire. This success lasted only 6 months, as the brain part that was worked on, came back and the cocain lust returned. (This is to show how the brain and cocain mix over a period of many months or years). This experiment needless to say became a failure. In my procedure it's important that the patient be in a sleeping state so that he or she does not feel anything of this new feeling as it should not be allowed to be dwelled on. On this sleeping patient a brain scan must monitor this brain to check after hours of treatment, for the electrical output of its parts, an EEG (electroencephalograph) can be used along with a CT or MRI for internal brain pictures, before and after. This tumor part of the brain may not be at all detected as a tumor unless a unusual electrical output is seen, meaning the patient is having an attack, which is not likely to occur while looking. When an electrical output is seen diminishing in a location as the procedure is proceeding then this location is where the tumor is at. Continue the treatment until there is no activity left in this sector and this tumor is dead and will not give the patient any more problems in there life time. Nor can it turn very bad and cause bigger problems as time goes on, which some times is the case. Vital signs must be watched for continuously, while performing this procedure, however nothing is likely to happen even in an uncontrolled environment due to the very tiny final chemically changed cocain of the evaporated piece used that is taken in one time only per session that lasts for approximately one half hour with no need to give more. If a patient has more than one tumor, you can take care of them, there and then, but better to schedule this for another time, as each tumor's requirements for self destruct is different. If done, this is coined by me as a brain sweep, which I suspect had other unusual good end results for me. At this point the treatment must stop when there is no electrical activity left in such a location. I might add that a brain scan can be done on me to locate this small dead area, where I believe my asthmatic tumor lies dead with no electrical activity there. Tumors located in other parts of the brain will cause other abnormalities such as epilepsy with involuntary muscle movements, and so on to other tumors located else where, causing their own unique problems depending on what net work they are connected to when an unwanted electric discharge comes about, which many times is unpredictable as to when it will occurs. At times it may be a fairly constant current, giving that person continuous unwanted movements, all having different medical names for each happening that are caused I believe by tumors. My belief is that most tumors come about, from genetic defects, as do asthmatic ones, and can certainly developed other ways at times, one way is from an auto accident causing injury to the head or the taking into ones body questionable environmental chemicals etc. The important thing then is to cure these tumors by self destruct, that was discovered by me quite by accident and to finally have put every thing together in my thinking on the subject due to enough layman's knowledge at my disposal, especially on the workings of the body. There is a possibility a cocaine solution can be put on a cancerous body spot and it might die with out using surgery or radiation with its bad side affects, if it is accessible. To be sure, a wide internal cancerous spread seems almost hopeless for the time being in curing. However my logic is, if the main ingredients in cocain that are responsible in killing brain tumors could be isolated, then this can lead to killing metastatic cancerous cells before doing damage to healthy organs, with no mutated changes to such cells as they are stopped cold by self destruct. This to me narrows the time factor in curing such wayward cells as they are especially sensitive to this new cocain's ingredients, after all these are tumors, even if its just a one cell tumor. A cocain use here may be different from my brain tumor cure, as it could require isolating these final changed chemicals in the evaporation of this substance into the developing of pills or intravenous of such ingredients with its own time, amount, and duration factors in performing this procedure for a cure. There is a strong possibility that such isolated main chemicals can cure tumors with out the patient feeling different there by allowing the patient to stay awake as the procedure is perform. The problem with a cancer cure lies in not knowing what drugs to work with on such tumors and then the constant mutation of such cells when using new man made drugs that at times just slowed down such tumors, only for them to mutate and continue on as if nothing happen. The only answer seems to be in killing them quickly, with mother nature's wonder drug in a systematic way. Developing only magic bullets from this changed substance could be elusive at first, after all you are dealing with non free base cocain and free base cocain which not only has many different derivatives but every thing can combine and change chemically in various complicated ways for a cure. I am sure in free basing, major chemical changes were made especially in the final evaporations process. Still this may not work on tumors in other parts of the body besides the brain, due to other parts of the body's unique resistance perhaps to this free base form, if so then such new main chemicals must be identified in the chemical vapors of this free based substance to be made into pill form or injected in a procedure that correlates the above for brain tumors. Rest to sure the cure is in cocain one way or another for tumors. Mother nature is best in knowing how to make drugs for cures, so much so that Marijuana is legal to use for patients in treating an eye disease called glaucoma by doctors so that the patient does not go blind, might not the same be done with my procedure in saving 5,000 lives a year in the United States alone from asthmatic attacks that do kill people, not to mention various other tumors that inflicted great sufferings to millions, and often times death to such people. SINCERELY YOURS, FRANK GULISANO ROBINHOODONE@AOL.COM

1. I claim by using mother nature's natural cocain as a finished product and freebasing it with baking soda (other agents that can be used are ammonia, and ether) which was used in the cooking process, except for ammonia, a product was arrived at that was vastly different chemically from the original cocain that now has a killing effect on tumors by disrupting its abnormal chemistry, when this new substance is vaporized by further heating it so that a final chemical change has taken place and taken in by a sedated patient it is instantly absorbed in the blood and reaches every where especially the brain in ridding it of tumors if they are there, by performing my procedure with its proper timing, duration and volume, and weight that is mention with the use of existing hospital equipment by qualified personnel. When every thing is working together a cure will come about! No surgery or radioactive material is needed for such a cure. Brain tumors are associated with different body diseases depending on what nerve net work in the body it has access to, directly or indirectly. Asthmatic tumors have access to the respiratory system in giving breathing problems. I have cured my asthma that I was born with, with my procedure approximately 15 years ago with no occurrences to date and claim it was a brain tumor in causing such difficulty in my breathing for 55 years. The cure for all tumors lies in the ingredients of cocain and its chemical changes that was brought about and must be applied in a systematic way to such tumors especially to metastasis tumors. If my procedure is not effective on other body tumors as it is on brain tumors then the magic bullet must be isolated from this cocain with its own injective procedure that will correlate my original procedure.
